(19) United States
(12) Patent Application Publication
(10) Pub. No.: US 2005/0031540 A1
(43) Pub. Date: Feb. 10, 2005

(54) Visco dye

(76) Inventor: Per Julius Nielsen, Viborg (DK)

Correspondence Address: NIXON PEABODY, LLP, 401 9TH STREET, NW, SUITE 900, WASHINGTON, DC, 20004-2128, US

(21) Appl. No.: 10/496,153

(22) PCT Filed: Nov. 20, 2002
(86) PCT No.: PCT/DK02/00780

(30) Foreign Application Priority Data
Nov. 20, 2001 (DK) PA 2001 01730

(51) Int. Cl. A61K 49/00

(52) U.S. Cl. 424/9.6

(57) ABSTRACT

There is disclosed a composition or kit for use in so-called viscodye enhanced ocular surgery, i.e. cataract surgery. The composition comprises a first viscoelastic substance containing a visualising agent (viscodye) used for staining the capsule of the crystalline lens prior to a Continious Curvilinear Capsulorhexis (CCC). A kit contains in addition a second clear viscoelastic agent for protection and expansion. The viscosity of the viscodye secures safe and precise application within the eye and the visualising agent can be used in such low concentrations, that the surgeon is allowed to see and work through it.

### BACKGROUND OF THE INVENTION

1. Field of the Invention

The present invention relates to a composition for use in intraocular surgery, preferably in cataract operations. Moreover the invention relates to a composition set and a kit for use in intraocular surgery, preferably in cataract operations.

2. Description of Related Art

The present invention is intended for cataract surgery, primarily where a Continious Curvilinear Capsulorhexis (CCC) is performed either anteriorly or posteriorly in the lens capsule. Though in the following, the invention will primarily be described in this light, it is within the scope of the invention to apply the composition, the composition set and the kit according to the invention in connection with other kinds of intraocular operations, i.e. such cases, whether it is traumatic or elective cataract surgeries, where the capsule of the lens need to be more clearly presented or in other types of ocular surgeries, where a vital dye could make a difference to the outcome of the operation by a better tissue presentation to the surgeon.

In the normal eye, the transparent and biconvex crystalline lens is located behind the iris and in front of the corpus vitreum. The crystalline lens is composed of a capsule that encompasses the lens substance, i.e. the lens epithelium, the cortex and the nucleus. A ring of zonular fibres that extend from the ciliary body to the anterior part of the lens capsule keeps the lens positioned within the eye. The capsule is an elastic type IV collagen basement membrane produced by the lens epithelial cells. Because of its transparency and because the refractive index equals that of the lens substance, the lens capsule cannot normally be visually discriminated from the lens substance during ocular surgery, except if a surgical microscope with an indirect or coaxial illumination is used. In such a situation, the clear capsule of the lens is presented as a dark reflex against the red fundus reflex created by the coaxial light or reflexes of light coming from the capsule when hit by the indirect light source.

Cataract is a common disease especially in elderly persons, where the lens substance gradually becomes less transparent. To restore vision, the optical pathway in the eye must be re-established. In cataract surgery this is done by removing the opaque lenticular material (nucleus and cortex) from within the lens capsular bag. To do this, an opening is needed in the lens capsule. This was initially performed by a so called “can-opener technique”, but now almost exclusively by a Continious Curviliniar Capsulorhexis (CCC), due to less risk of unwanted capsular tears with this. The established lens capsular bag is preserved to function as sack to keep an implanted artificial intraocular lens in place.

For cataract surgery today, the performance of a CCC is a key procedure that allows a safe phacoemulsification (removal of lens material) and prepares a safe implantation of an artificial lens within the capsular back created by first the CCC and the following phacoemulsification. The major reason to the importance and functionality of a CCC compared to a traditional “can opener capsulotomy” is, that a round continious opening of the lens capsule secures that elasticity of the collagen is preserved. This allows both stretch and manipulation during phacoemulsification and passage of greater issues, as i.e. an intraocular artificial lens through the round hole made by the CCC in the capsular bag. Therefore, the risk of making a tear in the capsular bag is less than with a “can opener technique”. In popular terms, a CCC is a peacemaker for the operation and any situation where it is not possible to perform a CCC carries a higher risk of complications during cataract surgery.

To visualise the lens capsule during the performance of a CCC, one needs a red fundus reflex produced by the coaxial light of the operating microscope. When retro-illumination is absent or reduced due to the low transparency accomplished by some cataracts, it may be difficult to discriminate the anterior capsule from the underlying lens tissue.

Inadequate visualisation of the capsule may accomplish a discontinuity or incomplete CCC with a high risk of tears towards or beyond the lens equator with associated complications. In order to minimise the risk for complications during cataract surgery, new techniques for CCC in special situations are continuously tried and steadily improved to increase the success and need to perform a CCC in almost every case. The ideal would be to find methods, that also could allow for a new surgeon to be taught a CCC with less risk of having complications during the learning period and to allow less experienced surgeons to have a higher success rate with a CCC and to allow all cataract surgeons to be able to perform a CCC in almost every case, both when a reduced or total loss of transparency of the anterior lens capsule is present or the surgeon is less experienced.

Increasing the visibility the lens capsule has been a difficult task in cataract surgery, especially where there is a partial or total loss of transparency as it is the case with a white or nearly white cataract. Much have been accomplished by better operating microscopes with ideal coaxial light sources, but in many places around the world the availability of such microscopes is reduced or cataracts with reduced transparency occur at a higher frequency due to postponed surgery and lack of surgical facilities and capacity with problems of visualisation of the lens capsule due to opacities in the lens even with a good operating microscope. Side-illumination, air, homologous blood and vital stains injected under the lens capsule or in the anterior eye chamber (flourocein, indocyan green, gentian violet, trypan blue) have all been tried with various success. Often, the techniques involved have been so complicated that a broader use especially in cases other than those with a total loss of transparency has not been advisable due also to difficulties with the availability, application, handling, control of and removal of the vital staining substance.

Only recently, the first commercial product to be used as an intraocular device for staining of the lens capsule using a vital dye was introduced. This product (Visionblue, Dorc) uses the vital stain trypan blue in a phosphate buffered basal salt solution. It has mostly an indication in totally white cataracts with no transparency at all and is used prior to a CCC to increase visibility of the lens capsule in the lack of a red reflex. Before the introduction of the dye in the eye, the anterior chamber is filled with air as a protective shield to the corneal endothelium and also to try and keep the stain in place with less possibility to escape the anterior lens capsule intended to be stained. However, both the interface created between air fluid and the injection speed created by the injected watery stain does, that an exact application directly the anterior lens capsule is made difficult. Due to this, also other structures besides the lens capsule is often stained. And in addition an uneven staining occurs of the lens capsule. Due to this uncontrollable staining, more and potentially toxic amounts of the stain is needed, than if the application could be made more precisely and ideally restricted to the area that needs staining. In addition, both vital stain and air needs to be removed and the chamber filled with a viscoelastic before a CCC can be performed and the surgery continued as a normal viscosurgical procedure.

However, the performance of a CCC is made much easier by the increased visibility created by an intensive blue staining of the lens capsule. The interior of the capsule seems to be more stainable than the lenticular mass (cortex and nucleus) as well as the outside of the capsule, thereby making a big and easily identifiable contrast between lenticular cortical material (only lightly stained) and the everted inside of the lenticular capsule (the internal surface and its monolayer of lens epithelial cells is heavily stained by the vital dye most likely due to an active uptake of stain.

Use of dyes in Capsulorhexis has been reported in Melles et al. “Trypan blue capsule staining to visualize the capsulorhexis in cataract surgery” in Journal of Cataract Refractive Surgery, Vol.25, January 1999, pages 7-9; Pandey et al. “Dye-enhanced cataract surgery” in Journal of Cataract Refractive Surgery, Vol.26, July 2000, pages 1052-1071; Pandey et al. “Staining the Anterior Capsule” in Journal of Cataract Refractive Surgery, Vol. 27, May 2001, pages 647-648; in European patent application EP 963 759 by Melles, and in International patent application WO01/03620 by Coroneo.

A drawback in using the above mentioned technique is that it involves an aqueous solution that needs to be injected in the anterior chamber filled with air. The injection is less controllable, painting less precise and the viscosurgical technique is interrupted creating a more difficult procedure than needed. Accordingly, it is difficult to place the vital dye exactly at the anterior surface of the lens capsule as the pressure from the piston of the syringe creates squirts by which the vital stain goes in all directions with inconsistent staining of the capsule and accidental staining of surrounding structures also.

Seeing that the composition, the composition set and the kit according to the present invention especially are suited for cataract surgery using a viscosurgical technique during the whole procedure and with no interruption, a description of such a surgical technique will be given in the following.

Most cataract surgeries are performed with local anaesthesia. Dilatation of the pupil is established prior to surgery with drops. In situations where injection or infiltration anaesthesia is preferred the injections are given from 10 and 20 minutes before surgery is begun.

The surgeon starts by making a small incision at the corneal margin through which incision the anterior chamber is filled with a viscoelastic substance to provide space and to protect intraocular structures especially the corneal endothelium.

A second incision is established at another position also at the corneal margin through which later phacoemulsification is done and the artificial lens implanted. First, however a Continious Curvilinear Capsulorhexis (CCC) is performed with a special forceps, where a puncture is made in the anterior capsule and extended peripherally and continued to perform a round opening centrally on the anterior surface of the lens. Through this opening the lens nucleus and the lens cortex are both removed with so-called phacoemulsification and irrigation/aspiration. This implies that the lens is split up into smaller pieces that are sucked out. The remaining capsule now appears as a sack suspended with thin threads (zonula threads). This sack is used as fixation of the new artificial lens in the posterior chamber of the eye right behind the pupil.

Immediately before implantation of the artificial lens, the sack is filled with a viscoelastic substance. After implantation of the lens, the viscoelastic substance is aspirated from the eye. The tightness of the wound is secured, eventually by use of a suture, and the operation is finished.

Viscoelastic substances such as hydroxypropyl cellulose, sodium hyaluronate, sodium chondroitin sulphate, or mixtures thereof are routinely used in intraocular surgery, i.e. cataract surgery. The viscoelastic substances may be of different molecular weight, concentration and viscosity. Different viscoelastics are commercially available for use in ocular surgery. Normally the viscoelastic is provided in a syringe with a cannula ready for use. The first commercial viscoelastic were a prescription drug, but today they have status as an intraocular device with a CE-mark registration only.

Using viscoelastic substances in connection with anaesthetics is known from International patent application WO 00/37047 by Nielsen. The use of dyes is not foreseen in that disclosure. A fluorescein dye in a viscoelastic substance also containing an anaesthetic is disclosed in British patent specification GB 1 113 760 by Krezanoski, where, however, staining of the surrounding tissue is unwanted. In this case, the fluorescent dye is used for its ability to make measurements of intraocular pressure with a so-called Goldmann Tonometer easier.

Using dyes for colouring a viscoelastic substance is generally known and reported in different documents. For example, a combination of a viscoelastic substance with a dye is disclosed in European patent application EP 1 132 065 by Melles, where the dye is used for colouring the viscoelastic substance to be able to distinguish this from for instance, the vitreous body. In International patent application WO 86/02548 by Malson, a composition is described for ophthalmology containing a polymeric dye in a concentration high enough for making the composition distinguishable from the surrounding tissue but low enough that a staining of the tissue is avoided if the dye should leak out of the coloured composition.

When several viscoelastic substances are used, it may be an advance to colour these substances different, which is reported in European patent application EP 974 320, where a tinting of the surrounding tissue is not desired. Dyes have also been used as a light absorbent in order to protect the eye against radiation, especially UV radiation during illumination, for example as disclosed in U.S. Pat. No. 5,008,102.

### SUMMARY OF THE INVENTION

It is a purpose of the present invention to provide a composition, a composition set and a kit for improvement of ocular surgery, especially such intraocular surgeries that involve a cataract extraction or lenticular surgery with a Continious Curviliniar Capsulorhexis.

This improvement is achieved in the present invention by a composition for use in intraocular surgery, preferably in dye enhanced cataract operations, comprising a first viscoelastic substance and a visualising agent, where the visualising agent in the composition is capable of staining eye tissue and intended for staining eye tissue and has a concentration sufficiently high to achieve staining during application of the composition in the eye.

The combination of a viscoelastic substance with a dye capable of and intended for staining has a great number of advantages as will be explained in more detail in the following. For example, by the invention, it is possible herewith to introduce a new entirety referred to as viscodye enhanced capsular (cataract) surgery. With this, there is no unnecessary interruption by introduction of air, no need for a high concentration of vital stain and a precise application and handling of the viscodye where it is needed and no need of removal until the end of surgery as is also the case with traditional viscosurgery.

Application of the composition according to the invention with a colored viscoelastic (viscodye) increases the precision of application of a vital dye to the exact site where it is supposed to stain underlying structures (i.e. lens capsule). This precision reduces the concentration of dye needed to color the capsule. At the same time this viscodye enhanced cataract surgery is meant to serve as part of a normal viscosurgical procedure involving the protective and expanding actions of the viscoelastic material while at the same time providing a staining of the lens capsule to help a CCC.

The composition set according to the present invention comprises a first composition as described above and furthermore it comprises a second composition with a viscoelastic substance without a visualising agent. In addition, the first and the second viscoelastic substance may be of different viscosities.

The kit according to the invention comprises a sterile packaged first syringe and a cannula containing a first composition comprising a first viscoelastic substance and a visualising agent, where the visualising agent in the composition is capable of staining eye tissue and intended for staining eye tissue and has a concentration sufficiently high to achieve staining during application of the composition in the eye, and a sterile packaged second syringe and a cannula containing a second composition comprising a second viscoelastic substance without a visualising agent, both ready for injection.

By using such a composition set and such a kit, it becomes possible to perform a viscodye enhanced ocular surgery with a secure application of the visualising agent/dye in the correct position, because the first viscoelastic substance may be applied exactly where it is needed, viz. just in front of the anterior capsule due to the gravity and viscosity of the substance. This allows the concentration of vital dye to be low as less dilution occurs and due to the possibility of an exact application. Uptake is taking place through the lens capsule by a specific and active staining of lens epithelial cells beneath the lens capsule. The important advantage of a low concentration of vital stain is sufficient visibility through the viscous dye, such that there is no need for interruption of the surgery for taking out the dye before the CCC is performed. The smaller concentration and exact application also reduces the risk of toxicity of the dye to other structures within the eye.

By eliminating the need for an air filled anterior chamber, the handling is made much easier for less trained surgeons and thereby creating a broader spectrum for dye enhanced cataract surgery. The viscodye itself or the kit involving a dye may also be combined with other materials/drugs, e.g. prophylactic antibiotic, in order to make cataract surgery procedures even more effective and adaptive to present or future situations, that may arise in and with new developments in ocular surgery.

Above all, the stability from the expansive and protective effect of the viscoelastic material in the eye is kept at all times, which is a major goal with viscosurgery. This greater stability of the eye during surgery using a viscodye opens the possibility for new and broader areas of application of dye enhanced cataract surgery due to its safety aspects, and it will not be restricted to difficult cases such as white cataracts alone. Alsol, it can be used as an educational tool in training of new and less experienced surgeons to master the essential step and important CCC procedure. The result is improved quality of eye surgery and last not least is a benefit for cataract patients.

Additionally, viscodye enhanced cataract surgery may also be helpful in cataract cases with a poor red light reflex secondary to other causes than lens opacity such as can be seen with a heavily pigmented fundus found in people with dark skin or pathology of the vitreous cavity (vitreous body).

When staining the anterior lens capsule with a low concentration viscodye, it has turned out that the dye actually is concentrated in and stains the lens epithelial cells sufficiently and more intensely than the surrounding tissue to allow for a safe CCC even in white cataracts where there is no red reflex. As the CCC is performed by turning the inside of the lens capsule out, this stands out with a greater contrast to the non-inverted capsule remaining. Even though the cells are arranged on the inside of the capsular bag in which the lens mass is situated, they have a higher affinity for the dye than surrounding structures inside and outside of the capsular bag and there seems to be no restriction to diffusion of the vital dye through the collagen capsular bag.

When the dye is added as a viscodye, viz. as a composition comprising a viscoelastic substance and a dye with a sufficient concentration, it is not only possible to achieve a staining in a very efficient manner, but the dye can be used in very low concentration, whereby it is possible to look through the dye and effectively make a CCC without first having to replace it by a clear viscoelastic.

Due to the easiness and precise application made possible by the viscosity of the viscodye, the present invention is suited for a broader and more safe use than vital stains based on an aqueous solution.

A staining with the use of the system according to the present invention might be effected according to two main principles.

A first principle could be denoted as a duovisc principle and the second principle could be denoted as a monovisc principle.

In the duovisc principle, the anterior chamber behind the cornea is filled with a viscoelastic composition having a relatively high viscosity. This viscoelastic composition is a clear transparent viscoelastica. The viscoelastic composition would fill out the chamber as a bolus. Between the lens and the bolus, another viscoelastic composition is added, namely, the first composition comprising a viscoelastic substance and a visualising agent (viscodye).

The viscodye would have a lower viscosity as compared to the viscosity of the viscoelastic composition constituting the bolus. This viscodye in a secure and efficient manner forms a thin and exact layer covering the anterior surface of the lens in the pupillary area and thereby effectively stains only the lens capsule.

The second viscoelastic agent with a higher viscosity provides expansion and protection of the inner structures of the eye as in normal viscosurgery and keeps the viscodye in place. After having provided the two compositions, it is possible to go directly to a CCC with a low concentration of vital dye or with a higher concentration simply aspirate the low viscosity viscodye before the performing the CCC and continuing viscosurgery.

With the monovisc principle, the anterior chamber is filled with a composition comprising a viscoelastic substance and a visualising agent, e.g. a vital dye (Viscodye). This viscodye both expands and protects the anterior chamber and provides vital staining of the anterior lens capsule without interrupting visualisation due to a barely visible concentration of dye. The viscosity is in this situation kept the same as in normal viscosurgery using only one viscoelastic. The CCC can be performed in the usual manner and surgery finished with viscodye staying for the rest of the procedure.

Both momovisc and duovisc principles are well known to cataract surgery and this invention introduces viscodye enhanced cataract surgery as a new principle in viscosurgery used for cataract.

Furthermore, it should be noted that the monovisc principle could be effected in one or more ways. Either a composition with a viscoelastic substance and a dye at a low concentration is used and maintained during the whole surgical procedure or the dye containing viscoelastic composition is used for providing the stain only at the beginning of surgery and if further viscoelastic is needed later during the procedure it may be changed to a clear viscoelastic after capsular staining has occurred, i.e. before lens implantation.

A disadvantage with the monovisc method compared to the duovisc principle is that the corneal endothelial cells also may take up some of the vital dye. This does not have a significant effect on visualisation, and i.e. trypan blue has earlier been used to stain and evaluate endothelial cell number in corneas prior to corneal transplants, and there seems to be a high tolerance and reversibility to vital staining and without a reduction in endothelial cell counts. The viscosity of the viscoelastic and the concentration of the dye may be changed according to the desired effects, either it is expansion and protection needing more viscosity or it is staining needing a higher concentration of vital stain.

Of cause, a viscodye also may also be used under the protection of air just to take advantage of the lower concentration and the more precise application presented by the viscosity of the substance. In such a case, the viscodye and the air needs to be aspirated and exchanged with a clear viscoelastic before the CCC and the viscosurgery can proceed.

In accordance with the present invention, it is possible to provide different embodiments of a set of compositions intended for use according to any of the vicodying principles.

Thus, it is possible to have a set of compositions for the duovisc principle containing a first composition with a first viscoelastic substance and a visualising agent and containing a second composition which comprises a second viscoelastic substance and which is transparent. The compositions are capable and intended to be used in contact with each other substantively without self-induced blending.

A second possible composition set to be used with the duovisc principle includes a first composition which has a first viscoelastic substance and a dye of low concentration as a visualising agent, where the visualising agent in the composition is capable of staining eye tissue and intended for staining eye tissue and still has a concentration sufficiently high to achieve staining during application of the composition in the eye. Further the set contains a second composition with a second viscoelastic substance, the composition being transparent without visualising agent. The second composition is used for substitution of the first composition.

If only one composition is used throughout the eye surgery, this composition would contain a visualising agent at a low concentration down to or less than 1 μg per litre, however still at a concentration such that the visualising agent in the composition is capable of staining eye tissue and intended for staining eye tissue and has a concentration sufficiently high to achieve staining during application of the composition in the eye.

In case that the monovisc principle is used in which the staining first composition is substituted by a non-staining second composition with viscoelastic substances, the concentration of the visualising agent in the first composition could be higher, e.g. up to 100 mg. per litre, in order to achieve a more intense staining. In this case, only a small amount of viscodye need to be applied in the pupillary area just above the lens capsule and air could be used as a protective shield. The following CCC is then done after aspirating the viscodye (and air if used) and filling the chamber with a bolus of a clear viscoelastic agent. A kit could also be used with this monovisc principle in which the transparent non-staining viscoelastic substance is included as part of a kit containing the viscodye.

A kit according to the present invention comprises two syringes, preferably where at least one or both syringes are having cannulas for injection.

Such type of kit could be suited for the duovisc principle. In this case, the first syringe contains a first composition with a viscoelastic substance and a visualising agent (viscodye) and the second syringe contains a second composition, which is different from the first composition because it has no stain in it.

As also been mentioned, it is preferred to provide a composition set for use in intraocular surgery. Such composition set make it possible to combine the advantages of using the composition according to the present invention with the advantages of using viscosurgery for the whole procedure and maybe combining it with prophylactic antibiotic.

The viscoelastic substance in any composition according to the invention or any composition contained in a composition set or kit according to the invention could involve sodium hyaluronate, which is known from viscosurgery. Typical concentrations are between 5 and 40 mg/ml, preferably between 10 and 20 mg/ml, and in Ringer lactate solution or BSS.

Any composition according to the invention or contained in a composition set or kit may preferably be pH-adjusted and balanced with respect to osmosis and salts for the purpose of avoiding a cytotoxic reaction or any discomfort to the patient when using it.

In a kit provided according to the invention, the syringes will visually differ from each other, so that the user does not mistake the syringes. The differentiation may be established by size, colour, shape, or in any other differentiable way.

The composition contained in each of the syringes may well be present without any preservation agents to avoid any toxic reaction to this inside the eye, especially from the corneal endothelium.

It is possible to use a number of vital dyes already known today, but also such which may become available in the future as the visualising agent. Thus, it is possible to make use of other vital dyes than trypan blue, seeing that a number of different vital dyes have been shown to be effective in staining the anterior capsule of the crystalline lens. These include azophlozin, basic blue, Bismarck brown, basis red, bengal red, brilliant cresyl blue, eosin, fluorescein, gentian violet, indocyanine green, Janus green, methylene green, methylene blue, neutral red and others. It has been found that of these, brilliant cresyl blue 1%, gentian violet 2%, methylene blue 5%, and trypan blue 0.1% stained the anterior capsule sufficiently to visualise the CCC in the absence of a red fundus reflex.

As mentioned earlier, it is possible, as compared to prior art, to make use of very much lower concentrations of dye in a composition according to the present invention. The concentration could be down to or even less than 1 μg/l corresponding to 0.000001 weight percent. Useable concentrations are for trypan blue 0.01%, for methylene rosanilin 0.001%, for methylene blue 0.5%, and for brilliant cresyl blue 0.1%. These concentrations are much lower compared with the concentrations used in other techniques making use of vital dyes to stain lens capsule or other ocular structures.

Possible viscoelastics are methylene hydroxypropylene cellulose, sodium hyaluronate, or sodium chondroitinsulphate, or mixtures thereof, or other viscoelastic substances for use in connection with intraocular operations. Concentrations between 5 and 40 mg/ml, preferably between 10 and 20 mg/ml, are preferred.

Short Description of the Drawing

In the following an explanation of the use of the system according to the present invention is given in connection with the drawing, wherein

### BRIEF DESCRIPTION OF THE DRAWINGS

FIG. 1 illustrates part of the eye,

FIG. 2 illustrates a monovisc principle, and

FIG. 3 illustrates a duovisc principle.

### DETAILED DESCRIPTION OF THE DRAWINGS

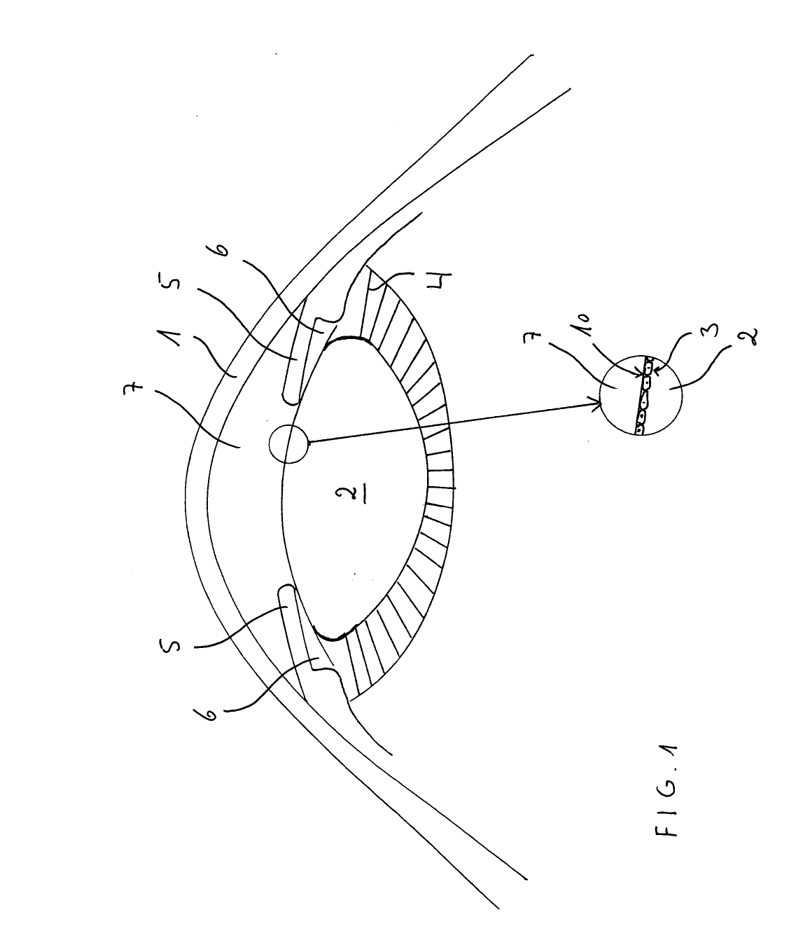

In FIG. 1, part of an eye is illustrated comprising a lens 2 surrounded by the pupil 5 and covered by the cornea 1. The volume between the lens 2 and the cornea 1 is denoted anterior chamber 7, whilst the volume between the pupil 5 and the lens 2 is called the posterior chamber 6. For illustration, part of the lens 2 is drawn enhanced in the lower part of the image showing in greater detail the epithelial cells 3 at the inner side of the capsule 10 of the lens 2 facing the anterior chamber 7. A ring of zonular fibres 4 that extend to the anterior part of the lens capsule 10 keeps the lens 2 positioned within the eye.

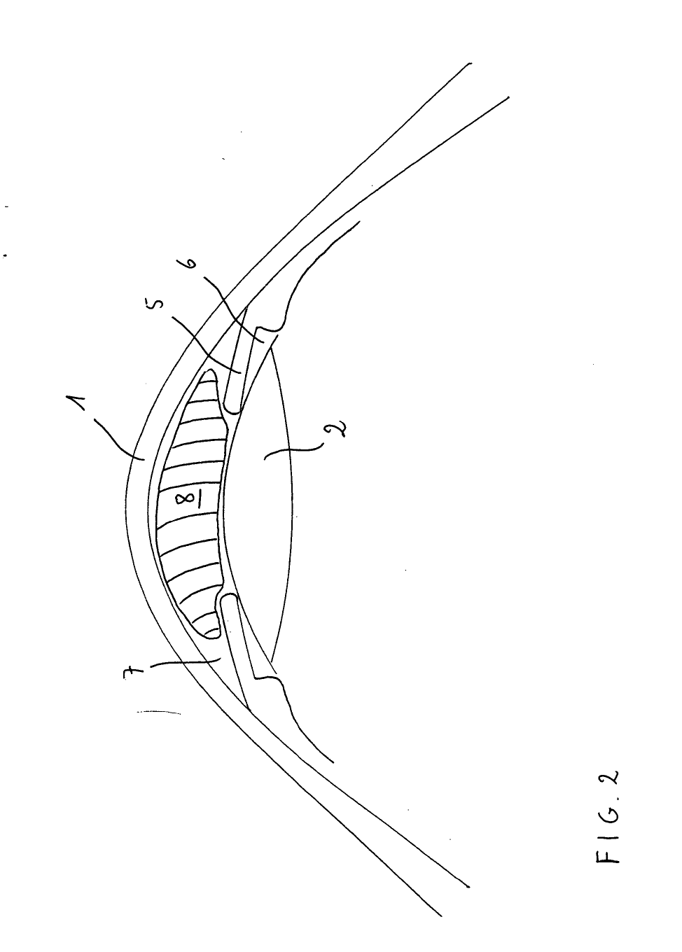

The anterior part of the eye is shown for the monovisc principle in FIG. 2. In this case, the anterior chamber 7 is filled with a bolus 8 of a viscodye composition according to the invention. The composition comprises a first viscoelastic substance and a visualising agent, where the visualising agent in the composition is capable of staining eye tissue and intended for staining eye tissue and has a concentration sufficiently high to achieve staining during application of the composition in the eye.

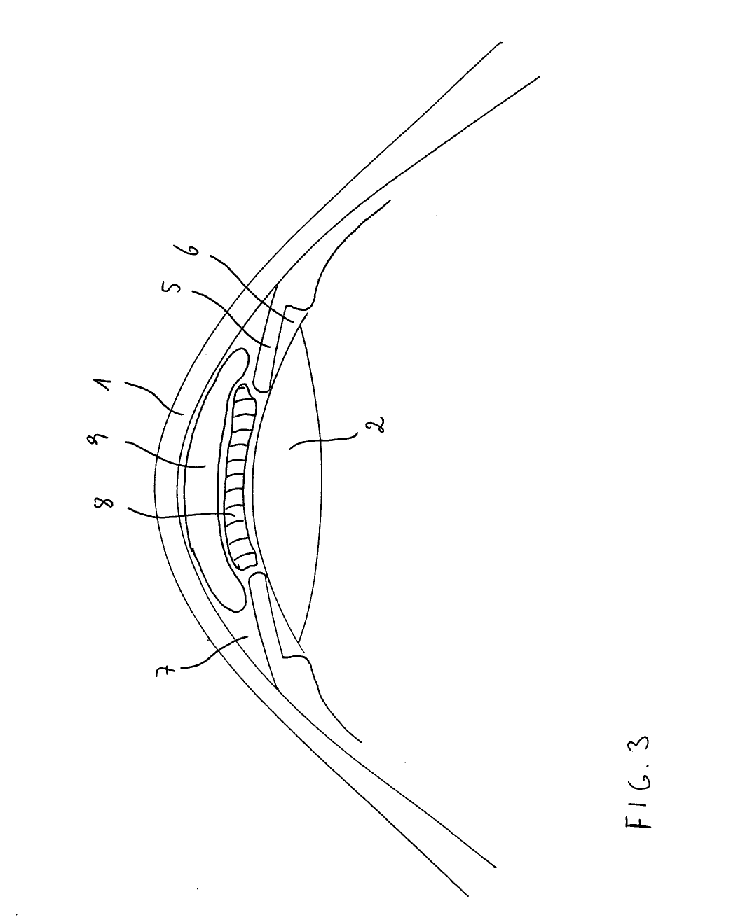

In FIG. 3, the duovisc principle is illustrated. In this case, a bolus 9 of a second composition is illustrated which contains a clear viscoelastic substance for protective and expansive reasons with a relatively high viscosity. The space between the lens 2 and the bolus 9 is occupied by the viscodye 8 exactly placed over the pupillary area close to the anterior part of the lens 2.

The visualising agent in the viscodye is provided in very low concentration to keep it highly transparent. The viscodye may stay there for the whole procedure or it may be exchanged for a clear viscoelastic either before or after the CCC, or a protective air exchange may be carried out before introducing the viscodye.

1. A composition set for use in intraocular surgery, preferably in dye enhanced cataract operations, which comprises a first composition with a first viscoelastic substance and a visualising agent, where the visualising agent in the composition is capable of staining eye tissue and intended for staining eye tissue and has a concentration sufficiently high to achieve staining during application of the composition in the eye, said composition set further comprises a second composition, said second composition being without visualising agent and comprising a second viscoelastic substance, wherein that the first composition is provided with a dye concentration low enough to keep the first composition highly transparent during application in the eye.

2. A composition set according to claim 1, wherein said first viscoelastic substance has a lower viscosity than said second viscoelastic substance.

3. A composition set according to claim 1, wherein said first viscoelastic substance has a concentration of between 5 and 40 mg/ml, preferably between 10 and 20 mg/ml, in Ringer lactate solution or basal salt solution (BSS), said first viscoelastic substance preferably comprising at least one from the group consisting of methylene hydroxypropylene cellulose, sodium hyaluronate, and sodium chondroitinsulphatesodium hyaluronate.

4. A composition set according to claim 1, wherein said visualising agent comprises at least one from the group consisting of azophlozin, basic blue, Bismarck brown, basis red, bengal red, brilliant cresyl blue, eosin, fluorescein, gentian violet, indocyanine green, Janus green, methylene green, methylene blue, neutral red and trypan blue or any known or future vital stains that could be used in the eye.

5. A composition set according to claim 1, wherein the concentration of said visualising agent in said composition is between 1 microgram per litre or less and 100 mg per litre.

6. A composition set according to claim 2, wherein the first and the second compositions are without preservations agents.

7. A composition set according to claim 2, wherein at least one of said first and said second viscoelastic substance has a concentration of between 5 and 40 mg/ml, and preferably between 10 and 20 mg/ml, in Ringer lactate solution or BSS, where said viscoelastic substances preferably comprises at least one from the group containing methylene hydroxypropylene cellulose, sodium hyaluronate, and sodium chondroitinsulphate or any known or future viscoelastic substances with a viscosity greater than water.

8. A composition set according to claim 2, wherein said first and said second composition are pH-adjusted and balanced with respect to osmosis and salt with the purpose of avoiding cytotoxic reactions and discomfort for the patient when using the compositions.

9. A composition set according to claim 2, wherein said first and said second composition are capable and intended to be used in contact with each other substantively without self-induced blending.

10. A kit for use in intraocular surgery, preferably in operations for cataract, comprising a first sterile packaged syringe containing a first composition comprising a first viscoelastic substance and a second sterile packaged syringe containing a second composition comprising a second viscoelastic substance, at least one or both said first and second syringe comprising a cannula ready for injection, wherein the first composition comprises a visualising agent (viscodye), where the visualising agent in the composition is capable of staining eye tissue and intended for staining eye tissue and has a concentration sufficiently high to achieve staining during application of the composition in the eye but low enough to keep the first composition highly transparent during application in the eye.

11. A kit according to claim 10, wherein the first and/or the second composition comprises a viscoelastic substance in concentrations of between 5 and 40 mg/ml, preferably between 10 and 20 mg/ml, in Ringer lactate solution or BSS, said viscoelastic substance preferably comprising at least one from the group consisting of methylene hydroxypropylene cellulose, sodium hyaluronate, and sodium chondroitinsulphatesodium hyaluronate or any known or future viscoelastic substances with a viscosity greater than water that can be used for eye surgery.

12. A method for performing eye surgery, the method comprising applying a first composition to the eye, said first composition comprising a first viscoelastic substance and a visualising agent, wherein the visualising agent in the first composition is capable of staining eye tissue and intended for staining eye tissue and has a concentration sufficiently high to achieve staining during application of the composition in the eye, wherein the method comprises application of a second composition with a second viscoelastic agent the second composition being applied as a bolus to the anterior chamber prior to application of the first composition, the first composition being added between the lens and the bolus, the second composition being a clear transparent viscoelastica having a higher viscosity than the first composition.

13. A method according to claim 12, wherein the first composition is applied for staining the anterior lens capsule.

14. A method according to claim 12, wherein the eye surgery comprises a capsulorhexis.

15. A method according to claim 12, wherein the first and/or the second composition comprises a viscoelastic substance in concentrations of between 5 and 40 mg/ml, preferably between 10 and 20 mg/ml, in Ringer lactate solution or BSS, said viscoelastic substance preferably comprising at least one from the group consisting of methylene hydroxypropylene cellulose, sodium hyaluronate, and sodium chondroitinsulphatesodium hyaluronate or any known or future viscoelastic substances with a viscosity greater than water that can be used for eye surgery.

16. A method according to claim 12, wherein said first viscoelastic substance has a lower viscosity than said second viscoelastic substance.

17. A method according to claim 12, wherein said visualising agent comprises at least one from the group consisting of azophlozin, basic blue, Bismarck brown, basis red, bengal red, brilliant cresyl blue, eosin, fluorescein, gentian violet, indocyanine green, Janus green, methylene green, methylene blue, neutral red and trypan blue or any known or future vital stains that could be used in the eye.

18. A method according to claim 12, wherein the concentration of said visualising agent in said composition is between 1 microgram per litre or less and 100 mg per litre.

19. A method according to claim 12, wherein the first and the second compositions are without preservations agents.

20. A method according to claim 12, wherein said first and said second composition are pH-adjusted and balanced with respect to osmosis and salt with the purpose of avoiding cytotoxic reactions and discomfort for the patient when using the compositions.

21. A method according to claim 12, wherein said first and said second composition are capable and intended to be used in contact with each other substantively without self-induced blending.

22. A kit according to claim 10, wherein said first viscoelastic substance has a lower viscosity than said second viscoelastic substance.

23. A kit according to claim 10, wherein said visualising agent comprises at least one from the group consisting of azophlozin, basic blue, Bismarck brown, basis red, bengal red, brilliant cresyl blue, eosin, fluorescein, gentian violet, indocyanine green, Janus green, methylene green, methylene blue, neutral red and trypan blue or any known or future vital stains that could be used in the eye.

24. A kit according to claim 10, wherein the concentration of said visualising agent in said composition is between 1 microgram per litre or less and 100 mg per litre.

25. A kit according to claim 10, wherein the first and the second compositions are without preservations agents.

26. A kit according to claim 10, wherein said first and said second composition are pH-adjusted and balanced with respect to osmosis and salt with the purpose of avoiding cytotoxic reactions and discomfort for the patient when using the compositions.

27. A kit according to claim 10, wherein said first and said second composition are capable and intended to be used in contact with each other substantively without self-induced blending.
